(19) United States
(12) Patent Application Publication
(10) Pub. No.: US 2012/0157799 A1
(43) Pub. Date: Jun. 21, 2012

(54) USING DEVICE BASED SENSORS TO CLASSIFY EVENTS AND GENERATE ALERTS

(76) Inventors: Abhilash Patangay, Inver Grove Heights, MN (US); Pramodsingh Hirasingh Thakur, White Bear Lake, MN (US); Yi Zhang, Plymouth, MN (US)

(21) Appl. No.: 13/331,288

(22) Filed: Dec. 20, 2011

Related U.S. Application Data
(60) Provisional application No. 61/424,989, filed on Dec. 20, 2010.

(51) Int. Cl. A61B 5/00; A61B 5/0205

(52) U.S. Cl. 600/301; 600/484

(57) ABSTRACT

The present subject matter includes apparatus, methods and device-readable media for using impedance and heart sounds to classify events and alerts. An apparatus can include a processor circuit configured to receive a physiological indication, classify the indication, and generate a multi-dimensional heart failure decompensation status indication. A method can include obtaining a physiological indication, classifying the indication and generating a multi-dimensional heart failure decompensation status indication. A device-readable medium can include instructions that, when performed by the device can obtain a physiological indication, classify the indication, and generate a multi-dimensional heart failure decompensation status alert.

### CLAIM OF PRIORITY

This application claims the benefit of priority under 35 U.S.C. §119(e) of Patangay et al., U.S. Provisional Patent Application Ser. No. 61/424,989, entitled “USING DEVICE BASED SENSORS TO CLASSIFY EVENTS AND GENERATE ALERTS”, filed on Dec. 20, 2010, which is herein incorporated by reference in its entirety.

### BACKGROUND

Implantable medical devices can obtain physiological indications associated with heart failure. In an example, such devices can be used to measure cardiac filling pressure, thoracic impedance, or cardiac output. In an example, such devices can be used to detect heart failure. Heart failure can be associated with weakened heart muscles and fluid accumulation in the lungs and elsewhere, among other things. Cho et al. U.S. Patent Publication No. 2010/0113888, entitled HEART FAILURE DECOMPENSATION DETERMINATION, refers to a method of detecting heart failure decompensation by sensing at least one physiological signal and comparing a heart failure variable with a threshold to detect a heart failure condition. (See Cho et al. at Abstract.) Sarkar et al. U.S. Patent Publication No. 2010/0030293, entitled USING MULTIPLE DIAGNOSTIC PARAMETERS FOR PREDICTING HEART FAILURE EVENTS, refers to techniques for using multiple physiological parameters to provide a warning for worsening heart failure using a medical device to detect a diagnostic parameter value, determine whether the value is outside the threshold zone and provide an alert upon the detection of worsening heart failure. (See Sarkar et al. at Abstract.)

### OVERVIEW

This document describes, among other things, an apparatus, method, and device-readable medium for using impedance and heart sounds to classify events and generate alerts. The present inventors have recognized, among other things, that a problem to be solved can include differentiating between cardiogenic heart failure events and non-cardiogenic heart failure events. This can be useful because treatment for cardiogenic events can be markedly different from treatment for non-cardiogenic events. In an example, the present subject matter can provide a solution to this problem, such as by using impedance and heart sounds to classify heart failure events and generate more useful alerts.

Example 1 includes an apparatus that can include a processor circuit configured to: receive an indication of cardiac filling pressure of a subject; receive an indication of thoracic fluid status of the subject; receive an indication of cardiac output of the subject; classify the indication of cardiac filling pressure into one of at least first and second cardiac filling pressure states; classify the indication of thoracic fluid status into one of at least first and second thoracic fluid status states; classify the indication of cardiac output into one of at least first and second cardiac output states; and generate a multi-dimensional heart failure decompensation status indicator using the classified indication of cardiac filling pressure, the classified indication of thoracic fluid status, and the classified indication of cardiac output.

In Example 2, the subject matter of Example 1 can optionally include a multi-dimensional heart failure decompensation status alert comprising separate cardiac filling pressure, thoracic fluid status, and cardiac output dimensions.

In Example 3, the subject matter of one or any combination of Examples 1-2 can optionally include an ambulatory device comprising the processor circuit, wherein the ambulatory device comprises a sensor configured to detect the indication of cardiac filling pressure of a subject, the indication of thoracic fluid status of the subject, and the indication of cardiac output of the subject.

In Example 4, the subject matter of one or any combination of Examples 1-3 can optionally include a sensor configured to detect one or any combination of the following cardiac filling pressure indications: a S3 heart sound amplitude, a pulmonary arterial pressure, left ventricular end diastolic pressure, or a left atrial pressure.

In Example 5, the subject matter of one or any combination of Examples 1-4 can optionally include a sensor configured to detect one or any combination of the following thoracic fluid status indications: thoracic impedance, a respiratory rate, a respiratory volume, a tidal volume, lung sound, or a lymphatic pressure or flow.

In Example 6, the subject matter of one or any combination of Examples 1-5 can optionally include a sensor configured to detect one or any combination of the following cardiac output indications: a core temperature of the subject, a peripheral temperature of the subject, a systolic time interval, a pre-ejection period, a S1 amplitude, a pulmonary artery pressure, or an intracardiac impedance.

In Example 7, the subject matter of one or any combination of Examples 1-6 can optionally include a processor circuit is configured to receive or classify an indication of respiratory function, and to generate the multi-dimensional heart failure decompensation status alert using the classified indication of respiratory function, the multi-dimensional heart failure decompensation status alert comprising a separate respiratory function dimension.

In Example 8, the subject matter of one or any combination of Examples 1-7 can optionally include an ambulatory device comprising the processor circuit, wherein the ambulatory device comprises a sensor configured to detect the indication of cardiac filling pressure of a subject, the indication of thoracic fluid status of the subject, the indication of cardiac output of the subject, and the indication of respiratory function.

In Example 9, the subject matter of one or any combination of Examples 1-8 can optionally include a processor configured to classify the indications of cardiac filling pressure, thoracic fluid status, and cardiac output into one of respective first and second states indicative of the severity of heart failure.

In Example 10, the subject matter of one or any combination of Examples 1-9 can optionally include a therapy control circuit configured to provide a control signal adapted to adjust, initiate, or cease a therapy regimen using the multi-dimensional heart failure decompensation indication.

In Example 11, the subject matter of one or any combination of Examples 1-10 can optionally include an electronic medical database or memory storage device capable of storing a history of at least one of (1) the indications of cardiac filling pressure, thoracic fluid status, and cardiac output; or (2) the multi-dimensional heart failure decompensation indication.

In Example 12, the subject matter of one or any combination of Examples 1-11 can optionally include a processor configured to compare at least one of the indications of cardiac filling pressure, thoracic fluid status, and cardiac output to information about at least a portion of the history.

In Example 13, the subject matter of one or any combination of Examples 1-12 can optionally include a processor configured to obtain an indication of cardiac filling pressure of the subject and classifying the subject as hemodynamically stable or hemodynamically unstable therefrom; obtain an indication of thoracic fluid status of the subject and classifying the subject as dry or wet therefrom; obtain an indication of cardiac output of the subject and classifying the subject as warm or cold therefrom.

Example 14 can include, or can optionally be combined with the subject matter of one or any combination of Examples 1-13 to include, subject matter (such as a method, a means for performing acts, or a machine-readable medium including instructions that, when performed by the machine, cause the machine to perform acts) comprising obtaining an indication of cardiac filling pressure of a subject; obtaining an indication of thoracic fluid status of the subject; obtaining an indication of cardiac output of the subject; classifying the indication of cardiac filling pressure into one of at least first and second cardiac filling pressure states; classifying the indication of thoracic fluid status into one of at least first and second thoracic fluid status states; classifying the indication of cardiac output into one of at least first and second cardiac output states; and generating a multi-dimensional heart failure decompensation status alert using the classified indication of cardiac filling pressure, the classified indication of thoracic fluid status, and the classified indication of cardiac output, the multi-dimensional heart failure decompensation status alert comprising separate cardiac filling pressure, thoracic fluid status, and cardiac output dimensions.

In Example 15, the subject matter of one or any combination of Examples 1-14 can optionally include instructions that, when performed by the device, comprise obtaining an indication of respiratory function; classifying the indication of respiratory function into one of at least first and second respiratory function states; and wherein the generating the multi-dimensional heart failure decompensation status alert comprises using the classified indication of respiratory distress, wherein the multi-dimensional heart failure decompensation status alert comprises a separate respiratory function dimension.

Example 16 can include, or can optionally be combined with the subject matter of one or any combination of Examples 1-15 to include subject matter (such as a method, a means for performing acts, or a machine-readable medium including instructions that, when performed by the machine, cause the machine to perform acts) comprising: obtaining an indication of cardiac filling pressure of a subject; obtaining an indication of thoracic fluid status of the subject; obtaining an indication of cardiac output of the subject; classifying the indication of cardiac filling pressure into one of at least first and second cardiac filling pressure states; classifying the indication of thoracic fluid status into one of at least first and second thoracic fluid status states; classifying the indication of cardiac output into one of at least first and second cardiac output states; and generating a multi-dimensional heart failure decompensation status indicator using the classified indication of cardiac filling pressure, the classified indication of thoracic fluid status, and the classified indication of cardiac output.

In Example 17, the subject matter of one or any combination of Examples 1-16 can optionally be performed such that the multi-dimensional heart failure decompensation status alert comprises using separate cardiac filling pressure, thoracic fluid status, and cardiac output dimensions.

In Example 18, the subject matter of one or any combination of Examples 1-17 can optionally be performed such that the classifying the indication of cardiac filling pressure comprises using one or any combination of the following indications: a S3 heart sound amplitude, a pulmonary arterial pressure, right ventricular pressure, left ventricular pressure, or a left atrial pressure.

In Example 19, the subject matter of one or any combination of Examples 1-18 can optionally be performed such that classifying the indication of thoracic fluid status comprises using one or any combination of the following indications: a thoracic impedance, a respiratory rate, a minute ventilation, a tidal volume, a lung sound, a rapid shallow breathing index, or a lymphatic pressure or flow.

In Example 20, the subject matter of one or any combination of Examples 1-19 can optionally be performed such that the classifying the indication of cardiac output comprises using one or any combination of the following indications: a core temperature of the subject, a peripheral temperature of the subject, a systolic time interval, a pre-ejection period, a S1 amplitude, a pulmonary artery pressure, or an intracardiac impedance.

In Example 21, the subject matter of one or any combination of Examples 1-20 can optionally be performed comprising: obtaining an indication of respiratory function; classifying the indication of respiratory function into one of at least first and second respiratory function states; and wherein the generating the multi-dimensional heart failure decompensation status alert comprises using the classified indication of respiratory distress, wherein the multi-dimensional heart failure decompensation status alert comprises a separate respiratory function dimension.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an illustration of portions of an example of a system that can use an implantable or other ambulatory device.

FIG. 2 is an illustration of portions of an example of a system that can use an implantable or other ambulatory device.

FIG. 3 is a block diagram illustrating portions of an example of an apparatus such as to classify a sensed or detected physiological indication.

FIG. 4 is an illustration of an example of a method of generating a multi-dimensional heart failure decompensation status alert.

FIG. 5 is an illustration of an example of a method of classifying a physiological indication into at least first and second states.

### DETAILED DESCRIPTION

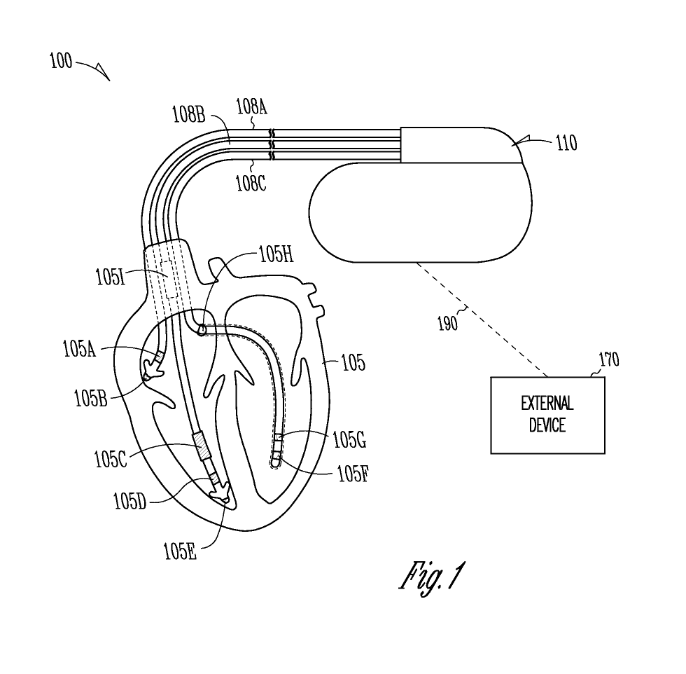

This document discusses, among other things, an apparatus, method, and device-readable media for using impedance and heart sounds to classify events and generate alerts. An ambulatory medical device includes medical devices that can be worn, implanted, or partially implanted. FIG. 1 illustrates generally an example of portions of an apparatus 100 that can enable detecting a physiological impedance and heart sounds and classifying alerts and events. In the example of FIG. 1, an ambulatory medical device, such as an implantable medical device (IMD) 110, can be configured to monitor or provide therapy to an ambulatory patient. The ambulatory medical device can include an external (e.g., wearable) medical device or an implantable medical device, among one or more other devices. For example, an ambulatory medical device can include a pacemaker, an implantable cardioverter defibrillator (ICD), a cardiac resynchronization therapy (CRT) device, a pulmonary artery (PA) pressure sensor, a neurostimulation device, a physiological signal monitor, a cardiovascular monitor, a stent, a drug pump, or a combination of these or other devices. In an example, the IMD 110 can be configured to sense physiological data, to derive physiological measures or correlations, or to store data such as for later communication or reference. Examples of physiological data can include implantable electrograms, surface electrocardiograms, heart rate intervals (e.g. AA, VV, AV, or VA intervals), electrogram templates such as for tachyarrhythmia discrimination, pressure (e.g., intracardiac or arterial pressure), oxygen saturation, heart rate variability, heart sounds, phonocardiograms, impedance, respiration, posture, intrinsic depolarization amplitude, lymphatic flow, temperature, or the like. The system can include an ambulatory programmer or other external device 170 that can communicate wired or wireless signals 190 with the ambulatory device, such as by using radio frequency (RF) or other telemetry signals.

While only one IMD is illustrated in FIG. 1, more than one IMD may be used. For example, multiple medical devices with specific functions may be used according to their respective functions. In an example, an IMD can include more than one device, with each device having one or more functions. Similarly, the position of the IMD 110 can vary, and the IMD 110 can be configured so as to accommodate one or more desired positions. Examples of other positions can include the patient's abdomen, back, or arm, among others.

The IMD 110 can be coupled by one or more leads 108A-C to the heart 105. Cardiac leads 108A-C can respectively include a proximal end that can be coupled to the IMD 110 and a distal end that can be coupled by electrical contacts or “electrodes,” such as 105A-I, to one or more desired locations, such as at one or more portions of a heart 105. In an example, one or more of the leads can deliver cardioversion, defibrillation, pacing, or resynchronization therapy, or a combination thereof to at least one chamber of the heart 105. In an example, one or more of the leads 108A-C can be electrically coupled to a sense amplifier to sense an electrical cardiac signal. In an example, the IMD 110 can include one or more extracardiac leads, such as a subcutaneous lead, a sub-pectoral lead, or a epicardial lead. Although a specific arrangement of leads and electrodes is depicted in the illustration, the present methods and systems will work in a variety of configurations and with a variety of leads.

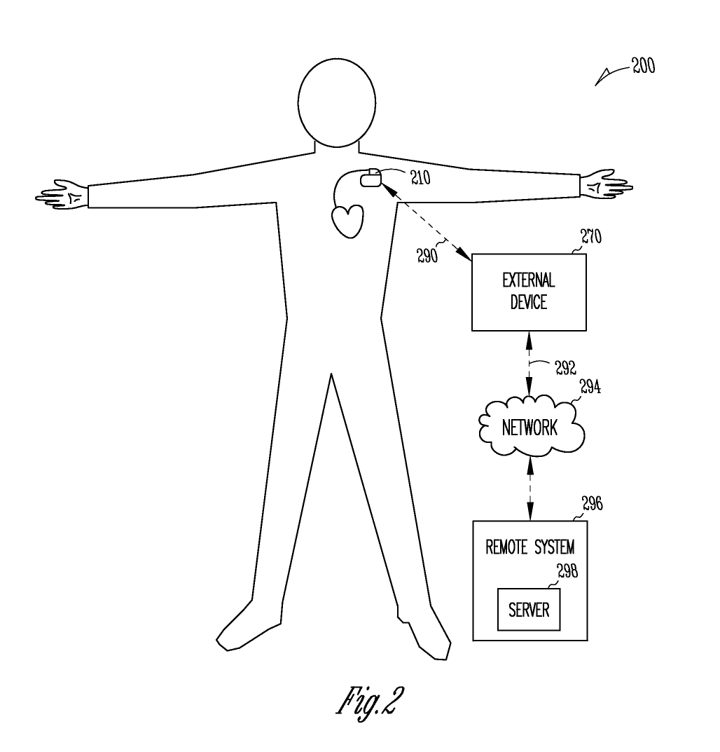

FIG. 2 illustrates generally an example of portions of an apparatus 200 including an IMD 210. In an example, the IMD 210 can be configured to be capable of bidirectional communication 290 with an external device 270. Examples of the bidirectional communication can include radio frequency (RF), Bluetooth, ultrasonic, infrared, or another communication connection. In an example, a local interface can include a device configured to receive input, process instructions, store data, present data in a human-readable form, or communicate with one or more other devices. In an example, the IMD 210 can receive commands from an external device 270 such as a local interface. The IMD 210 can be configured to communicate, such as via a wired or wireless communication link 290, one or more patient indications to the external device 270. Examples of patient indications can include one or more sensed or derived indications such as heart rate, heart rate variability, S3 heart sound, systolic timing interval, pre-ejection period, S1 amplitude, pulmonary arterial pressure, left atrial pressure, right ventricular pressure, left ventricular pressure, left ventricular end diastolic pressure, intracardiac impedance, thoracic impedance, minute ventilation, tidal volume, rapid shallow breathing index, respiration rate, lymphatic pressure, lymphatic flow, lung sounds, temperature, cardiac contractility, patient position and posture, autonomic balance, activity, motor trends, therapy history, heart rate variability trends or templates, or one or more trends, templates, or abstractions derived from sensed physiological data. Patient indications can include or be derived from one or more physiological indications, such as the physiological data described above, among others. The IMD 210 can also be configured to communicate one or more device indications to an external device 270. Examples of device indications can include lead/shock impedance, pacing amplitudes, pacing capture thresholds, or one or more other device metrics. In an example, the IMD 210 can be configured to communicate sensed physiological signal data to the external device 270, which can communicate 292 the signal data, such as via a network 294 to a remote system 296 such as for processing. In an example, when more than one IMD 210 has been employed, the multiple IMD 210 devices can be configured to communicate with each other, such as by using the communication link 290.

In an example, the external system 270 can include a local interface. The local interface can be located near the patient. The local interface can be attached, coupled, integrated or incorporated with a personal computer or a specialized device, such as a medical device programmer. The local interface can include a hand-held device, such as a personal digital assistant (PDA), smart phone, personal computer, or any specialized device. The local interface can be configured to communicate with a remote system 296. Examples of a remote system 296 include a remote computer or server or the like. The communication link between the local interface and the remote interface can be made through a computer or telecommunications network 294. Examples of the network 294 can include one or more wired or wireless networks such as the Internet, satellite telemetry, cellular or other mobile telephone telemetry, microwave telemetry, or using one or more other long-range communication networks. In some examples, the remote system can include a server 298.

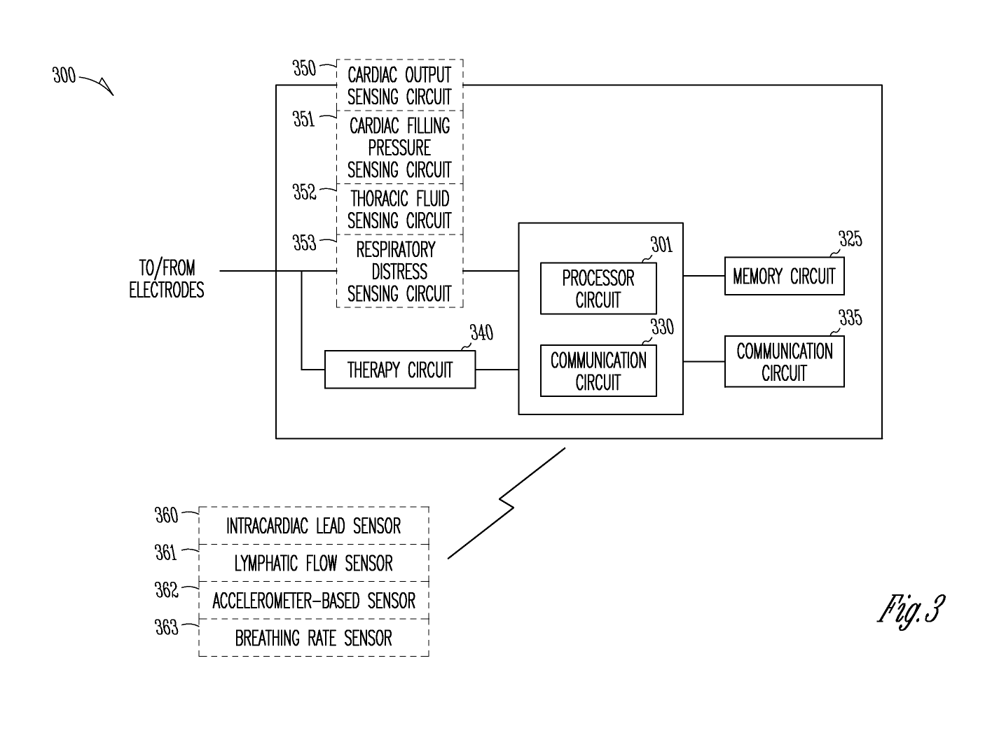

FIG. 3 illustrates generally an example of portions of an example of the apparatus 300. In the example of FIG. 3, the apparatus 300 can include a processor circuit 301 configured to receive one or more physiological indications, such as an indication of one or a combination of the following: cardiac filling pressure, thoracic fluid status, cardiac output, or respiratory function. In an example, the processor circuit 301 can perform instructions, such as for obtaining a physiological indication, classifying the physiological indication, and generating a multi-dimensional heart failure decompensation status alert using the classified physiological indication. For example, the instructions can include classifying the physiological indication into at least first and second states.

In an example, the apparatus can include a processor circuit 301, such as for selectively connecting to one or more sensors. The one or more sensors can be located on the leads 108A-C, within a housing of an electronics unit, or elsewhere. In an example, the sensor can be configured to detect an indication of cardiac filling pressure, an indication of thoracic fluid status, or an indication of cardiac output. Examples of sensors that can be used to detect one or more physiological indications can include, but are not limited to, one or more of: an electrical cardiac signal sensing circuit, a heart sounds sensor, a transthoracic impedance measurement circuit, an intracardiac impedance measurement circuit, an intravascularly-located pressure sensor, a minute ventilation or tidal volume or breathing rate or other respiratory sensor, a lymphatic pressure sensor, a lymphatic flow sensor, an accelerometer such as for sensing physical activity, posture, heart sound, sleep, lung sound, or another parameter, a blood pressure sensor, a wall motion sensor, or a heart rate variability sensor.

In an example, a cardiac output sensing circuit 350 can be selectively coupled to a sensor 360 that can be configured to detect an indication of cardiac output of the subject, such as one or more of: a core temperature of the subject, a peripheral temperature of the subject, a systolic time interval, a pre-ejection period (PEP), a S1 heart sound amplitude, a pulmonary artery pressure (PAP), or an intracardiac impedance. In an example, the indication of cardiac output or stroke volume can be extracted from an indication of one cardiac cycle of intracardiac impedance, such as by comparing the impedance signal (Z) maxima and minima (maxZ−minZ) or by analyzing the location of the maximum amplitude of the first derivative of the impedance signal (dZ/dT). In an example, indication of cardiac output can be extracted from an indication of peak to peak impedance change during systole of the RV to Can intracardiac impedance, or coronary venous pressure.

In an example, a cardiac filling pressure sensing circuit 351 can be selectively coupled to a sensor 361 configured to detect an indication of cardiac filling pressure of the subject. In an example of a sensor selectively configured to detect an indication of cardiac filling pressure, the sensor can detect an indication including, but not limited to, one or more of: a S3 heart sound amplitude, a pulmonary arterial pressure, a left atrial pressure, right ventricular pressure, or central venous pressure.

In an example, a thoracic fluid sensing circuit 352 can be selectively coupled to a sensor 362 that can be configured to detect an indication of thoracic fluid status of the subject. In an example in which the sensor 362 includes a thoracic impedance or like sensor 362 that is selectively configured to detect an indication of thoracic fluid, the sensor 362 can also detect an indication including, but not limited to, one or more of: thoracic impedance, lung sound, a respiratory rate, a minute ventilation, or a tidal volume. In an example, the sensor 362 can be configured to detect a lymphatic pressure or flow.

In an example, a respiratory distress sensing circuit 353 can be selectively coupled to a thoracic impedance or other sensor 363 that can be configured to detect an indication of respiratory distress. In an example in which the sensor is selectively configure to detect an indication of respiratory distress, the sensor can detect an indication including, but not limited to, one or more of: breathing rate, breath-to-breath interval, tidal volume, breath-to-breath interval variation, sleep disordered breathing index, period breathing index, respiratory rate at rest, tidal volume at a particular posture, or minute ventilation during different levels of activities.

In an example, a processor circuit 301 can include a discrimination circuit that detect abnormal patterns in one of more of the physiological indications, such as by comparing the one or more of the physiological indications to a respective specified threshold value. For example, the processor circuit 301 can be configured to classify the indication of cardiac filling pressure into one of at least first and second cardiac filling pressure states, to classify the indication of thoracic fluid status into one of at least first and second thoracic fluid status states, to classify the indication of cardiac output into one of at least first and second cardiac output states, or to classify an indication of respiratory function into one of at least first and second respiratory function states. In an example, the processor circuit can be configured to classify a physiological indication into one of the first and second states that can indicate the severity of heart failure. In an example, the processor 301 can be configured to compare at least one of the physiological indications (e.g., of cardiac filling pressure, thoracic fluid status, cardiac output, or another indication) to similar historical information from the same patient or one or more similar patients.

In an example, the processor circuit 301 can generate a multi-dimensional heart failure decompensation status alert using (1) a classified indication of cardiac filling pressure, (2) a classified indication of thoracic fluid status, and (3) a classified indication of cardiac output. The multi-dimensional heart failure decompensation status alert generated by the processor circuit 301 can include separate cardiac filling pressure, thoracic fluid status and cardiac output dimensions. In another example, the processor circuit 301 can generate a multi-dimensional heart failure decompensation status alert additionally using (4) a classified indication of respiratory function and having a separate (e.g., fourth) respiratory function dimension.

In an example, the apparatus can include a therapy circuit 340 such as can be selectively coupled to one or more of various sensors 360-363. The apparatus can include a therapy control circuit configured such as to provide a control signal that can be configured to adjust, initiate, or cease a therapy regimen using the multi-dimensional heart failure decompensation status indication. In an example, a therapy circuit 340 can include therapy energy generation circuitry (e.g., capacitive, inductive, or other) such as for generating, storing or delivering an electrostimulation, cardioversion, defibrillation, drug delivery, or other energy.

In an example, one or more physiological sensing circuits 350-353 can be communicatively coupled to the processor circuit 301. The processor circuit 301 can include a processor such as a microprocessor, a digital signal processor, application specific integrated circuit (ASIC), microprocessor, or other type of processor, such as for interpreting or executing instructions in software or firmware. The processing circuit 301 can include one or more other circuits or sub-circuits, such as to perform the functions, methods, or techniques described herein. In an example, the ambulatory device 110 can include multiple processor circuits 301. One more processor circuits 301 can be included in one or more ambulatory devices 110. The processor circuit 301 can include a communication circuit 330 to communicate information with a communication circuit of a second device 335. In some examples, the second device 335 can include a display such as for communicating information about the multi-dimensional heart failure decompensation status indication to a user.

The processor circuit 301 can be communicatively coupled to a memory circuit 325. In an example, one or more physiological indications received by the processor circuit can be stored such as by a memory circuit 325. In an example, the classified indications are stored for subsequent display by a memory circuit 325. In an example, the apparatus can be coupled to an electronic medical record database or memory storage device capable of storing one or more of the indications of cardiac filling pressure, thoracic fluid status or cardiac output. In an example, the apparatus can be coupled to an electronic medical record database or memory storage device capable of storing the multi-dimensional heart failure decompensation indication.

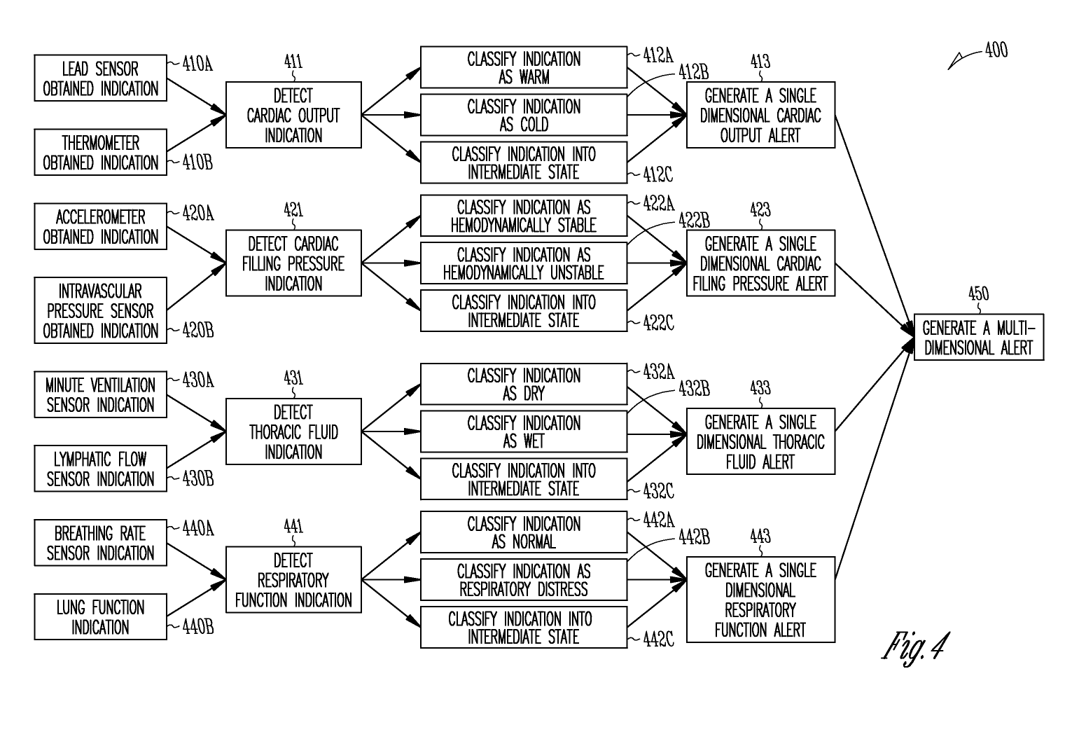

FIG. 4 illustrates generally an example of a technique 400 that can include providing a multi-dimensional heart failure decompensation status indication using multiple physiological indications. At 410A-B, information (e.g., an accelerometer obtained indication or a thermometer obtained indication, or both) can be obtained from one or more implantable sensors, such as described above with respect to FIG. 3. At 411, a subject's indication of cardiac output can be detected, such as by the one or more implantable sensors. At 411, the indication of cardiac output used for classification can include one or any combination of the following: a core temperature of the subject, a peripheral temperature of the subject, a systolic time interval, a pre-ejection period, a S1 amplitude, a pulmonary artery pressure, an intracardiac impedance, or another indication of cardiac output.

At 412A-C, the indication of cardiac output can be used to classify the subject into a particular state of a plurality of states. For example, the indication of cardiac output can be used for classification into one of at least first and second states. At 412A, the indication of cardiac output can be used for classification into a first state, such as “warm,” which can correlate with normal cardiac output. At 412B, the indication of cardiac output can be used for classification into a second state such as “cold,” which can correlate with decreased cardiac output. At 412C, the indication of cardiac output can be used for classification into an intermediate state, such as a state that is between “warm” and “cold.” At 413, such classification can be used to determine a cardiac output alert, which can be provided to a caregiver or other user or to an automated process, such as a “single-dimensional” alert.

At 420A-B, information (e.g., an accelerometer obtained indication or an intravascular pressure sensor obtained indication or both) can be obtained from one or more implantable sensors, such as described above with respect to FIG. 3. At 421, a subject's indication of cardiac filling pressure can be detected, such as by the one or more implantable sensors. At 421, the one or more indications of cardiac filling pressure used for classification can include one or any combination of the following: a S3 heart sound amplitude, a pulmonary arterial pressure, a right ventricular pressure, a left ventricular pressure, or a left atrial pressure, or another indication of cardiac filling pressure.

At 422A-C, the indication of cardiac filling pressure can be used to classify the subject into a particular state of a plurality of states. For example, the indication of cardiac filling pressure can be used to classify the indication into one of at least first and second states. At 422A, the indication of cardiac filling pressure can be used to classify the indication into a first state, such as “hemodynamically stable,” which can correlate with normal cardiac filling pressure. At 422B, the indication of cardiac filling pressure can be used for classification into a second state, such as “hemodynamically unstable,” which can correlate with increased cardiac filling pressure. At 422C, the cardiac filling pressure-related indication can be used to classify a subject into an intermediate state, such as can be between the hemodynamically stable and unstable states. At 423, such classification can be used to generate a cardiac filling pressure alert, which can be provided to a caregiver or other user or to an automated process, such as a “single-dimensional” alert.

At 430A-B, information (e.g., a thoracic impedance sensor indication or a lymphatic flow sensor indication) can be obtained from one or more implantable sensors, such as described above with respect to FIG. 3. At 431, an indication of thoracic fluid status can be detected, such as by the one or more implantable sensors. At 431, the indication of thoracic fluid status used for classification can include one or any combination of the following: a thoracic impedance, a lung sound, a respiratory rate, a minute ventilation, a tidal volume, a rapid shallow breathing index, or a lymphatic pressure or flow, or another indication of thoracic fluid status.

At 432A-C, the indication of thoracic fluid status can be used to classify the subject into a particular state of a plurality of states. For example, the indication of thoracic fluid status can be used to classify the indication into one of at least first and second states. At 432A, the indication of thoracic fluid status can be used to classify the indication into a first state such as “dry,” which can correlate with normal thoracic fluid. At 432B, the indication of thoracic fluid status can be used to classify the indication into a second state such as “wet,” which can correlate with worsening pulmonary edema or pulmonary congestion. At 432C, the indication of thoracic fluid status can be used to classify a subject into an intermediate state, such as can be between the “wet” and “dry” states. At 433, such classification can be used to generate a thoracic fluid alert, which can be provided to a caregiver or other user or to an automated processes, such as a “single-dimensional” alert.

At 440A-B, information (e.g., a breathing rate sensor indication or a lung function indication) can be obtained from one or more implantable sensors, such as described above with respect to FIG. 3. At 441, a subject's respiratory function indication can be detected, such as by the one or more implantable sensors. At 441, the one or more respiratory function indications that can be used for classification can include one or any combination of the following: fast respiratory rate, increased breathing effort, increased sleep disordered breathing index, increased minute ventilation at a particular activity level, cyanosis, unusual posturing, tachycardia, a change in mental state due to hypoxemia, or another respiratory function indication.

At 442A-C, the respiratory function indication can be used to classify the subject into a particular state of a plurality of states. For example, respiratory function indication can be used to classify the indication into one of at least first and second states. At 442A, the respiratory function indication can be used to classify the indication into a first state, such as “normal.” At 442B, the respiratory function indication can be used to classify the indication into a second state such as “respiratory distress.” At 442C, the respiratory function indication can be used to classify a subject into an intermediate state, such as can be between the “normal” and “respiratory distress” states. At 443, such classification can be used to generate a respiratory function alert, which can be provided to a caregiver or other user or to an automated process, such as a “single-dimensional” alert.

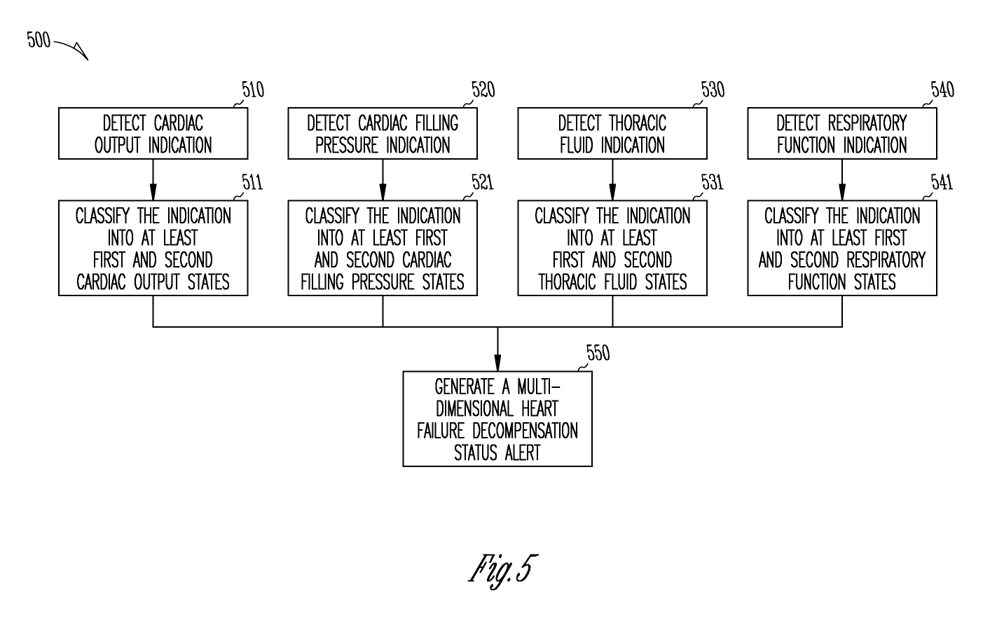

At 450, a multi-dimensional heart failure decompensation status alert can be generated (e.g., as discussed in the example of FIG. 5). At 450, the technique can include generating a multi-dimensional heart failure decompensation status alert using the classified indication of cardiac output 412A-C, the classified indication of cardiac filling pressure 422A-C, and the classified indication of thoracic fluid status 432A-C. At 450, the multi-dimensional heart failure decompensation status alert can include a separate cardiac output dimension 413, a cardiac filling pressure dimension 423, and a thoracic fluid status dimension 433. At 450, the technique can include generating a multi-dimensional heart failure decompensation status alert using the classified indication of respiratory function 442A-C. At 450, the multi-dimensional heart failure decompensation status alert can include a separate respiratory function dimension 443.

FIG. 5 illustrates generally an example of a technique 500 that can include classifying physiological indications to generate a multi-dimensional heart failure decompensation status alert. At 510, an indication of cardiac output can be obtained using information provided by a sensor. At 511, the indication of cardiac output can be used to classify the subject into one of at least first and second cardiac output states. At 511, the indication of cardiac output can be classified as “cold” when (1) the peripheral temperature measurement of the subject decreases by a specified amount when compared to the core temperature measurement of the subject, (2) when a S1 amplitude indication decreases below a specified threshold, (3) when a S1-S2 interval shortens below some threshold value, or (4) when the pre-ejection period lengthens beyond a specified threshold. At 511, the indication of cardiac output can be classified as “warm” when the indication is within a specified normal range. At 511, the indication of cardiac output can be classified into an intermediate state when an obtained indication falls outside of the specified normal range and does not meet the specifications for classification as “cold.” The classification of the indication of cardiac output can occur from one or any combination of indications of cardiac output.

At 520, an indication of cardiac filling pressure can be obtained using information provided by a sensor. At 521, the indication of cardiac filling pressure can be classified into one of at least first and second cardiac filling pressure states. At 521, the classification of the indication of cardiac filling pressure can occur such as by classifying the indication as “hemodynamically unstable” when (1) the S3 heart sound amplitude indication increases by at least 50%, (2) when pulmonary arterial pressure exceeds some threshold value, or (3) when the left ventricular end diastolic pressure exceeds a specified threshold. At 521, the indication of cardiac filling pressure can be classified as “hemodynamically stable” when one or more obtained physiological indications or cardiac filling pressure are within specified normal ranges. For example, the indication of cardiac filling pressure can be classified into an intermediate state when the indication falls outside the specified normal range and does not reach the specified threshold for classification as “hemodynamically unstable.” The classification of the indication of cardiac filling pressure can occur from one or any combination of indications of cardiac filling pressure.

At 530, an indication of thoracic fluid status can be obtained using information provided by a sensor. At 531, the indication of thoracic fluid status can be classified into one of at least first and second thoracic fluid status states. At 531, the indication of thoracic fluid status can be classified as “wet” when tidal volume measurements of the subject fall below a specified threshold, thoracic impedance falls below a specified threshold, lung sounds increase above a specified threshold, when lymph flow increases above some specified threshold value, or when the subject is in respiratory distress. At 531, the indication of thoracic fluid status can be classified as “dry” when a physiological indication is within a specified normal range. In an example, the indication of thoracic fluid status can be classified into an intermediate state when an indication falls outside the specified normal range but does not meet the threshold requirement for classification as “wet.” The classification of the indication of thoracic fluid can occur from one or any combination of indications of thoracic fluid status.

At 540, an indication of respiratory function can be obtained using information provided by a sensor. At 541, the indication of respiratory function can be classified into one of at least first and second respiratory function states. At 541, when a respiratory rate increase above a specified threshold is obtained, the indication can be classified as indicative of “respiratory distress.” At 541, the indication of respiratory function can be classified as “normal” when a physiological indication is within a specified normal range. In an example, the indication of respiratory rate can be classified into an intermediate state when the indication of respiratory function does not meet the criteria for “normal” or “respiratory distress.” The classification of respiratory function can occur from one or any combination of indications of respiratory function.

At 550, a multi-dimensional heart failure decompensation status alert can be generated. At 550, a multi-dimensional alert can be created by obtaining any two or more single dimensional alerts. At 550, a multi-dimensional alert can include obtaining an indication of cardiac filling pressure of a subject and classifying the subject as “hemodynamically stable” or “hemodynamically unstable” therefrom, obtaining an indication of thoracic fluid status of the subject and classifying the subject as “dry” or “wet” therefrom, and obtaining an indication of cardiac output of the subject and classifying the subject as “warm” or “cold” therefrom. At 550, the multi-dimensional alert can include obtaining an indication of respiratory function and classifying the indication as indicative of respiratory distress or normal therefrom. At 550, the multi-dimensional alert can be created such as by classifying the subject as “hemodynamically stable,” “dry,” and “warm;” as “hemodynamically unstable,” “dry,” and “warm;” as “hemodynamically stable,” “wet,” and “warm;” as “hemodynamically unstable,” “wet,” and “warm;” as “hemodynamically stable,” “dry,” and “cold;” as “hemodynamically unstable,” “dry,” and “cold;” as “hemodynamically stable,” “wet,” and “cold;” or as “hemodynamically unstable,” “wet,” and “cold.” At 550, the multi-dimensional alert can include an indication of respiratory distress. At 550, the multi-dimensional alert can be created using information about one or more of the intermediate states described above.

### Additional Notes

An implantable ambulatory device can include one or more of the features, structures, methods or combinations thereof described herein. For example, an implantable or other ambulatory device can be implemented to include one or more of the advantageous features or processes described below. It is intended that the apparatus, method and device-readable media need not include all of the features described herein, but may be implemented to include selected features that provide for unique structures or functionality. Such an apparatus, method, and device-readable media may be implemented to provide a variety of therapeutic or diagnostic functions.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An apparatus comprising:
a processor circuit configured to:
receive an indication of cardiac filling pressure of a subject;
receive an indication of thoracic fluid status of the subject;
receive an indication of cardiac output of the subject;
classify the indication of cardiac filling pressure into one of at least first and second cardiac filling pressure states;
classify the indication of thoracic fluid status into one of at least first and second thoracic fluid status states;
classify the indication of cardiac output into one of at least first and second cardiac output states; and
generate a multi-dimensional heart failure decompensation status indicator using the classified indication of cardiac filling pressure, the classified indication of thoracic fluid status, and the classified indication of cardiac output.

2. The apparatus of claim 1, wherein the multi-dimensional heart failure decompensation status alert comprises separate cardiac filling pressure, thoracic fluid status, and cardiac output dimensions.

3. The apparatus of claim 1, comprising an ambulatory device comprising the processor circuit, wherein the ambulatory device comprises a sensor configured to detect the indication of cardiac filling pressure of a subject, the indication of thoracic fluid status of the subject, and the indication of cardiac output of the subject.

4. The apparatus of claim 3, wherein the sensor is configured to detect one or any combination of the following cardiac filling pressure indications: a S3 heart sound amplitude, a pulmonary arterial pressure, left ventricular end diastolic pressure, or a left atrial pressure.

5. The apparatus of claim 3, wherein the sensor is configured to detect one or any combination of the following thoracic fluid status indications: thoracic impedance, a respiratory rate, a respiratory volume, a tidal volume, lung sound, or a lymphatic pressure or flow.

6. The apparatus of claim 3, wherein the sensor is configured to detect one or any combination of the following cardiac output indications: a core temperature of the subject, a peripheral temperature of the subject, a systolic time interval, a pre-ejection period, a S1 amplitude, a pulmonary artery pressure, or an intracardiac impedance.

7. The apparatus of claim 1, wherein the processor circuit is configured to receive or classify an indication of respiratory function, and to generate the multi-dimensional heart failure decompensation status alert using the classified indication of respiratory function, the multi-dimensional heart failure decompensation status alert comprising a separate respiratory function dimension.

8. The apparatus of claim 7, comprising an ambulatory device comprising the processor circuit, wherein the ambulatory device comprises a sensor configured to detect the indication of cardiac filling pressure of a subject, the indication of thoracic fluid status of the subject, the indication of cardiac output of the subject, and the indication of respiratory function.

9. The apparatus of claim 1, wherein the processor is configured to classify the indications of cardiac filling pressure, thoracic fluid status, and cardiac output into one of respective first and second states indicative of the severity of heart failure.

10. The apparatus of claim 1, wherein the apparatus includes a therapy control circuit configured to provide a control signal adapted to adjust, initiate, or cease a therapy regimen using the multi-dimensional heart failure decompensation indication.

11. The apparatus of claim 1, wherein the apparatus is coupled to an electronic medical database or memory storage device capable of storing a history of at least one of (1) the indications of cardiac filling pressure, thoracic fluid status, and cardiac output; or (2) the multi-dimensional heart failure decompensation indication.

12. The apparatus of claim 3, wherein the processor is configured to compare at least one of the indications of cardiac filling pressure, thoracic fluid status, and cardiac output to information about at least a portion of the history.

13. The apparatus of claim 1, wherein the processor is configured to
obtain an indication of cardiac filling pressure of the subject and classifying the subject as hemodynamically stable or hemodynamically unstable therefrom;
obtain an indication of thoracic fluid status of the subject and classifying the subject as dry or wet therefrom;
obtain an indication of cardiac output of the subject and classifying the subject as warm or cold therefrom.

14. A device-readable medium including instructions that, when performed by the device, comprise:
obtaining an indication of cardiac filling pressure of a subject;
obtaining an indication of thoracic fluid status of the subject;
obtaining an indication of cardiac output of the subject;
classifying the indication of cardiac filling pressure into one of at least first and second cardiac filling pressure states;
classifying the indication of thoracic fluid status into one of at least first and second thoracic fluid status states;
classifying the indication of cardiac output into one of at least first and second cardiac output states; and
generating a multi-dimensional heart failure decompensation status alert using the classified indication of cardiac filling pressure, the classified indication of thoracic fluid status, and the classified indication of cardiac output, the multi-dimensional heart failure decompensation status alert comprising separate cardiac filling pressure, thoracic fluid status, and cardiac output dimensions.

15. A device-readable medium of claim 14, wherein the instructions that, when performed by the device, comprise:
obtaining an indication of respiratory function;
classifying the indication of respiratory function into one of at least first and second respiratory function states; and
wherein the generating the multi-dimensional heart failure decompensation status alert comprises using the classified indication of respiratory distress, wherein the multi-dimensional heart failure decompensation status alert comprises a separate respiratory function dimension.

16. A method comprising:
obtaining an indication of cardiac filling pressure of a subject;
obtaining an indication of thoracic fluid status of the subject;
obtaining an indication of cardiac output of the subject;
classifying the indication of cardiac filling pressure into one of at least first and second cardiac filling pressure states;
classifying the indication of thoracic fluid status into one of at least first and second thoracic fluid status states;
classifying the indication of cardiac output into one of at least first and second cardiac output states; and
generating a multi-dimensional heart failure decompensation status indicator using the classified indication of cardiac filling pressure, the classified indication of thoracic fluid status, and the classified indication of cardiac output.

17. The method of claim 16, wherein the multi-dimensional heart failure decompensation status alert comprises using separate cardiac filling pressure, thoracic fluid status, and cardiac output dimensions.

18. The method of claim 16, wherein the classifying the indication of cardiac filling pressure comprises using one or any combination of the following indications: a S3 heart sound amplitude, a pulmonary arterial pressure, right ventricular pressure, left ventricular pressure, or a left atrial pressure.

19. The method of claim 16, wherein the classifying the indication of thoracic fluid status comprises using one or any combination of the following indications: a thoracic impedance, a respiratory rate, a minute ventilation, a tidal volume, a lung sound, a rapid shallow breathing index, or a lymphatic pressure or flow.

20. The method of claim 16, wherein the classifying the indication of cardiac output comprises using one or any combination of the following indications: a core temperature of the subject, a peripheral temperature of the subject, a systolic time interval, a pre-ejection period, a S1 amplitude, a pulmonary artery pressure, or an intracardiac impedance.

21. The method of claim 16, comprising:
obtaining an indication of respiratory function;
classifying the indication of respiratory function into one of at least first and second respiratory function states; and
wherein the generating the multi-dimensional heart failure decompensation status alert comprises using the classified indication of respiratory distress, wherein the multi-dimensional heart failure decompensation status alert comprises a separate respiratory function dimension.
